(19) United States
(12) Patent Application Publication
(10) Pub. No.: US 2002/0002136 A1
(43) Pub. Date: Jan. 3, 2002

(54) Salts of glutathione

(76) Inventor: Rolland F. Hebert, Seattle, WA (US)

Correspondence Address: ROLLAND HEBERT, 427 BELLEVUE AVE E. SUITE 301, SEATTLE, WA, 98102, US

(21) Appl. No.: 09/893,762

(22) Filed: Jun. 27, 2001

Related U.S. Application Data
(60) Provisional application No. 60/214,573, filed on Jun. 28, 2000.

(51) Int. Cl. A61K 38/06; A61K 31/722

(52) U.S. Cl. 514/18; 514/55

(57) ABSTRACT

Stable salts of glutathione with polycations such as chitosan are described. The salts according to the invention are valuable for use as active constituents in pharmaceutical as well as cosmeceutical compositions.

### BACKGROUND-CROSS-REFERENCES TO RELATED APPLICATION

This application claims the benefit of Provisional Patent Application Ser. No.: 60/214573 filed on Jun. 28, 2000.

### FIELD OF THE INVENTION

The present invention relates to new water-soluble salts of glutathione.

1. Technical Field

This patent relates to new salts of glutathione with polycations, the processes for obtaining them and to therapeutic uses of these new salts. More particularly, the invention relates to salts deriving from the reaction between glutathione, s-nitroso-glutathione, and monoesters of glutathione and polycations such as chitosan, their production process, and pharmaceutical compositions that contain them as active principles.

2. Background of the Invention

Glutathione (GSH) is a naturally occurring tripeptide that has utility as a free radical scavenger. It is an important antioxidant and an essential cofactor for antioxidant enzymes. Glutathione is found in all animals, plants and microorganisms. Glutathione exists in two forms: the antioxidant reduced glutathione called GSH and the oxidized form, glutathione disulfide, GSSG.

Glutathione exists intracellularly primarily in its reduced form. In normal cells, the oxidized form of glutathione rarely exceeds 10% o the total glutathione concentration. (Kosower NS, Kosower EM. The glutathione status of cells. Intl Rev Cytology 1978; 54:109-156.) Glutathione is synthesized mainly in the liver in mammals and it is involved in three major functions in mammalian physiology: a) as a cofactor in the detoxification pathways; b) as a substrate for gamma-glutamyl transpeptidases; and c) for direct free-radical scavenging and as an antioxidant cofactor. Glutathione is essential for survival of the organism. Scientific literature on genetic GSH depletion in humans and animals indicates that such depletion results in pathology. (Meister A, Larsson A. Glutathione synthetase deficiency and other disorders of the gamma-glutamyl cycle. In: Scriver CR, et al eds. The Metabolic and Molecular Bases of Inherited Disease (Volume 1). New York: McGraw-Hill; 1995; 1461-1495 (Chapter 43); Beutler E. Nutritional and metabolic aspects of glutathione. Annu Rev Nutr 1989; 9:287-302.)

Glutathione is important in protein synthesis, cell maturation, intermediary metabolism, enzyme catalysis, transmembrane transport, and receptor action. Glutathione, as a reducing agent, is used to fine tune the redox state of cellular environments. (Meister A. Mitochondrial changes associated with glutathione deficiency. Biochim Biophys Acta 1995; 1271:3542.)

Metabolic oxidative processes generate large amounts of free radicals that result in endogenous oxidative stress. Superoxide, peroxide hydroxyl radical and other free radicals resulting from such processes are very highly reactive and can threaten the stability and integrity of biomolecules such as DNA, RNA and other proteins. Glutathione can be used to quench such free radicals.

Oxidative stress that has its origins outside of the body (exogenous oxidative stress) is an unfortunate result of living in the modern world. Thousands of toxic chemicals are found in the environment and these chemicals are sources of free radicals or other oxidant chemicals. Examples of such chemicals abound. However, the following exogenous oxidative stressors are the most important for this discussion: cigarette smoke, pharmaceutical products, halogenated hydrocarbons, dietaty factors, and ionizing radiation among others. Cigarette smoke contains many different chemicals and one puff of smoke contains trillions of free radicals. (Cross C E, Halliwell B, Borish E T, et al. Oxygen radicals and human disease (proceedings of a conference). Ann Intern Med 1987;107:526-545.) The smoke depletes vitamins C and E that are antioxidants. The cigarette tars are free radical generators with long half-lives and they are potent carcinogens. (Kidd P. The free radical oxidant toxins of polluted air. In: Levine S A, Kidd P M. Antioxidant Adaptation: Its Role in Free Radical Pathology. San Leandro, C A: Biocurrents; 1985:69-103.) Many drugs have oxidant properties and are capable of depleting liver, kidney, and heart GSH. (Hoyumpa A M, Schenker S. Drugs and the liver. In: Maddrey W C, ed. Gastroenterology and Hepatology: The Comprehensive Visual Reference. Philadelphia: Current Medicine; 1996:6.1-6.22.) The halogenated hydrocarbons are potent oxidants and are found everywhere. They are used in the plastics industry, as pesticides and herbicides and as propellants. Halocarbons contaminate a large portion of the ground water in the US. (Kidd P. The free radical oxidant toxins of polluted air. In: Levine S A, Kidd P M. Antioxidant Adaptation: Its Role in Free Radical Pathology. San Leandro, C A: Biocurrents; 1985:69-103.)

Dietary deficiencies of methionine, an essential amino acid and precursor of GSH can cause GSH depletion. (Mandl J, Banhegyi G, Kalapos M P, et al. Increased oxidation and decreased conjugation of drugs in the liver caused by starvation. Altered metabolism of certain aromatic compounds and acetone. Chem Biol Interact 1995; 96:87-101.) Ionizing radiation such as from X-rays or ultraviolet light from the sun can also cause GSH depletion. (Biaglow J E, Varnes M E, Epp E R, et al. Role of glutathione and other thiols in cellular response to radiation and drugs. Drug Metab Rev 1989;20:1-12).

Glutathione deficiencies can result in chronic diseases. Lack of GSH is contributory to liver injury and to an increase in morbidity associated with hypofunction of the liver. (Lomaestro B M, Malone M. Glutathione in health and disease: pharmacotherapeutic issues. Annals Pharmacother 1995;29: 1263-73.) In patients suffering from cirrhosis of the liver, abnormally low GSH plasma concentrations have been observed. (Chawla R K, Lewis F W, Kutner M H, et al. Plasma cysteine, cystine, and glutathione. Gastroenterology 1984; 87:770-776.) In a larger study of patients with cirrhosis, a four to eight fold decrease in GSH was seen. (Loguercio C, Delvecchio Blanco C, Coltorti M, et al. Alteration of erythrocyte glutathione, cysteine, and glutathionesynthetase in alcoholic and nonalcoholic cirrhosis. Scand J Clin Lab Invest 1992; 52:207-213.) Other studies have shown that plasma and liver GSH is decreased in patients with acute viral hepatitis and in cases of chronic hepatitis, alcohol liver disease or cirrhosis not related to alcohol. (Shigesawa T, Sato C, Marumo F. Significance of plasma glutathione determination patients with alcoholic and non-alcoholic liver disease. J Gastroenterol Hepatol 1992; 7:7-11. Seifert C F, Anderson DC, Bui B, et al. Correlation of acetaminophen and ethanol use, plasma glutathione concentrations and diet with hepatotoxicity. Pharmacotherapy 1994; 14:376-377.)

Lung tissue is at risk from oxidative stressors such as smoking, atmospheric pollutants and other types of inhaled environmental toxins. (Kidd P. The free radical oxidant toxins of polluted air. In: Levine S A, Kidd P M. Antioxidant Adaptation: Its Role in Free Radical Pathology. San Leandro, C A: Biocurrents; 1985:69-103.) GSH deficiencies have been shown in the following lung diseases: acute respiratory distress syndrome, asthma, chronic obstructive pulmonary disease, cystic fibrosis, and idiopathic pulmonary fibrosis. (Lomaestro B M, Malone M. Glutathione in health and disease: pharmacotherapeutic issues. Annals Pharmacother 1995;29: 1263-73; Pacht E R, Timerman A P, Lykens M G, et al. Deficiency of alveolar fluid glutathione in patients with sepsis and the adult respiratory distress syndrome. Chest 1991; 100:1397-1403).

Cells associated with the immune system are GSH dependent. Proliferation, growth and differentiation are all GSH dependant. Patients with immune defects have been shown to have low lymphocyte GSH levels. (Kinscherf R, Fischbach T, Mihim S, et al. Effect of glutathione depletion and oral N-acetyl-cysteine treatment on CD4+ and CD8+ cells. FASEB J 1994; 8:448-451.) The ability of the immune cells to respond is dependent upon the intracellular levels of GSH. Lowered GSH levels result in decreased ability of the immune cells to respond appropriately to challenge. (Droge W, Schulze-Osthoff K, Mihm S, et al. Functions of glutathione and glutathione disulfide in immunology and immunopathology. FASEB J 1994;8:1131-1138.) Chronic viral infections such as HIV and hepatitis C can lead to a decreased intracellular concentration of GSH in immune cells. (Anderson ME. Glutathione and glutathione delivery compounds. Adv Pharmacol 1997;3 8:65-78. Droge W, Gross A, Hack V, et al. Role of cysteine and glutathione in HIV infection and cancer cachexia: therapeutic intervention with N-acetylcysteine. Adv Pharmacol 1997; 38:581-600.)

The nervous system is also vulnerable to GSH depletion since it is a highly oxigenated system resulting in free radical production. Certain regions of the brain are particularly susceptible to GSH depletion resulting in tissue degeneration. One such region is the substantia nigra responsible for dopamine production. Parkinson's disease is caused by the decreased production of dopamine in the substantia nigra of the brain. Depleted GSH has been reported in patients with Parkinson's disease. (Adams J D Jr, Klaidman L K, Odunze I N, et al. Alzheimer's and Parkinson's Disease. Brain levels of glutathione, glutathione disulfide, and vitamin E. Mol Clin Neuropathol 1991; 14: 213-226. Jenner P. Oxidative damage in neurodegenerative disease. Lancet 1994(September 17);796-798.) Patients with Down's Syndrome (Trisomy 21) are known to have increased systemic oxidative stress. (Levine S A, Kidd P M. Antioxidant Adaptation: Its Role in Free Radical Pathology. San Leandro, C A: Biocurrents;1985:171-218. Alzheimer's patients have decreased GSH levels in cortical areas (Lohr J B, Browning J A. Free radical involvement in neuropsychiatric illnesses. Psychopharmacol Bull 1995;31:159-165. Jenner P. Oxidative damage in neurodegenerative disease. Lancet 1994(September 17);796-798.) GSH levels are also lower in the hippocampus a site primarily noted for short term memory initiation. (Adams J D Jr, Klaidman L K, Odunze I N, et al. Alzheimer's and Parkinson's Disease. Brain levels of glutathione, glutathione disulfide, and vitamin E. Mol Clin Neuropathol 1991; 14:213-226.) Diabetic neuropathy is associated with decrease levels of GSH. Supplementation with a GSH precursor, N-acetyl-cysteine resulted in the reversal of some of the neuropathy symptoms in an experimental diabetes animal model (Sagara M, Satoh J, Wada R, Yagihashi S, Takahashi K, Fukuzawa M, Muto G, Muto Y, Toyota T. Inhibition of development of peripheral neuropathy in streptozotocin-induced diabetic rats with N-acetylcysteine. Diabetologia 1996 March; 39(3):263-9.)

Glutathione levels are decreased with advancing age. Blood GSH concentrations of health young subjects (20-39 years of age) compared to healthy elderly subjects (60-79 years of age) showed that the young subjects had an increase of 17% blood levels of GSH as compared to the older subjects. (Lang C A, Naryshkin S, Schneider D L, et al. Low blood glutathione in healthy aging adults. J Lab Clin Med 1992; 120:720-725.) Interestingly, in another study, higher levels of GSH were correlated with good health, irrespective of age; subjects suffering from chronic disease had lower GSH mean concentrations as compared to those who were disease free. (Julius M, Lang C A, Glieberman L, et al. Glutathione and morbidity in a community-based sample of elderly. J Clin Epidemiol 1994; 47:1021-1026.)

Glutathione also functions as a carrier for nitric oxide, an important molecule in diverse physiological processes. Thus glutathione as s-nitroso-glutathione (an S-nitrosothiol) plays a role in cellular and tissue physiology be delivering appropriate amounts of nitric oxide to tissues. Glutathione, in its role as a nitric oxide donor, may be used for the treatment or prevention of disorders associated with relation of smooth muscle, such as airway obstruction, and other respiratory disorders, bladder dysfunction, premature labor and impotence (erectile dysfunction). Additionally, it may be used to alleviate smooth muscle contraction and spasm, and thus facilitate procedures involving diagnostic instrumentation, such as endoscopy, bronchoscopy, laparoscopy and cystoscopy. S-nitrosothiols also increase the binding affinity between hemoglobin and oxygen, and therefore, may be used to improve hemoglobin-oxygen binding, and oxygen transport to bodily tissues. Further, it may be used to inhibit contraction of skeletal muscles.

Methods of increasing cellular glutathione are needed in view of the potential beneficial effects on mammalian health. Glutathione levels can be increased by oral administration of glutathione. (Hunjan M K, Evered D F. Absorption of glutathione from the gastrointestinal tract. Biochim Biophys Acta 1985;815:184-188.) They can also be increased by administration of monoesthers of glutathione. Glutathione is a strong reducing agent, so that autooxidation occurs in the presence of oxygen or other oxidizing agents. New salts of glutathione are needed to address this issue as well as to address the need for new glutathione derivatives for prevention and treatment of conditions previously described.

3. Prior Art

Demopoulos, U.S. Pat. No. 6,159,500 et al. Dec. 12, 2000 Pharmaceutical preparations of glutathione and methods of administration thereof discloses the use of ascorbic acid to stabilize glutathione but does not disclose or teach the use of a polycation to stabilize glutathione. Jones et al. Jan. 11, 2000 U.S. Pat. No. 6,013,632 Compounds and their combinations for the treatment of influenza infection disclose the use of glutathione and its disulfide dimer for the treatment as well as prevention of influenza virus. However, this patent does not disclose or teach the use of a polycation to stabilize glutathione. Crystal, Nov. 23, 1999 U.S. Pat. No. 5,989,521 Method for augmenting a decreased level of reduced glutathione in the lung, discloses the use of glutathione to increase glutathione in the lung but does not disclose nor teach the use of a polycation to stabilize glutathione. Ohlenschlager, Feb. 29, 2000 U.S. Pat. No. 6,030,950 Pharmaceutical therapeutic use of glutathione derivative discloses the use of an acetyl derivative of glutathione but does not disclose nor teach the use of a polycation to stabilize glutathione.

Accordingly, there is need in the art for new, more stable glutathione salts as well as methods related to the use of such salts. There is also a need in the art for synthetic routes to make such new salts. The author of this present invention fulfills these needs.

### OBJECTS AND SUMMARY OF THE PRESENT INVENTION

It is an object of the present invention to provide safe, inexpensive, chemically stable salts of glutathione, monoesters of glutathione and s-nitrosoglutathione. It is also an object of the present invention to provide synthetic processes for the manufacture of these new salts of glutathione.

Briefly stated, the present invention accomplishes these objectives by disclosing new salts of glutathione, n-nitroso-glutathione and monoesthers of glutathione, methods for the use thereof and synthetic methods for their preparation. These new salts of glutathione of this present invention have utility in increasing blood and other tissue or fluid levels of glutathione, as well as treating or preventing a wide variety of conditions related to the aforementioned mechanisms of action of glutathione. Thus in one embodiment, a new glutathione salt is administered to a warm-blooded animal in need thereof In yet a further embodiment, a new glutathione salt is administered to a warm blooded animal to prevent and or treat the following conditions: aging of the skin, cancer, HIV, lung disease, diabetes, macular degeneration, asthma, atherosclerosis, Parkinson's disease, Alzheimer's disease, wound healing, inherited GSH deficiency conditions, conditions related to excessive expression of reactive oxygen species, liver disease, hepatitis, cirrhosis of the liver, viral infections, and conditions related to decreased NO levels. Other aspects of the present invention will become evident upon reference to the following detailed description.

### BRIEF DESCRIPTION OF THE DRAWINGS

FIG. 1 is an HPLC analysis of one of the salts of glutathione made according to this invention.

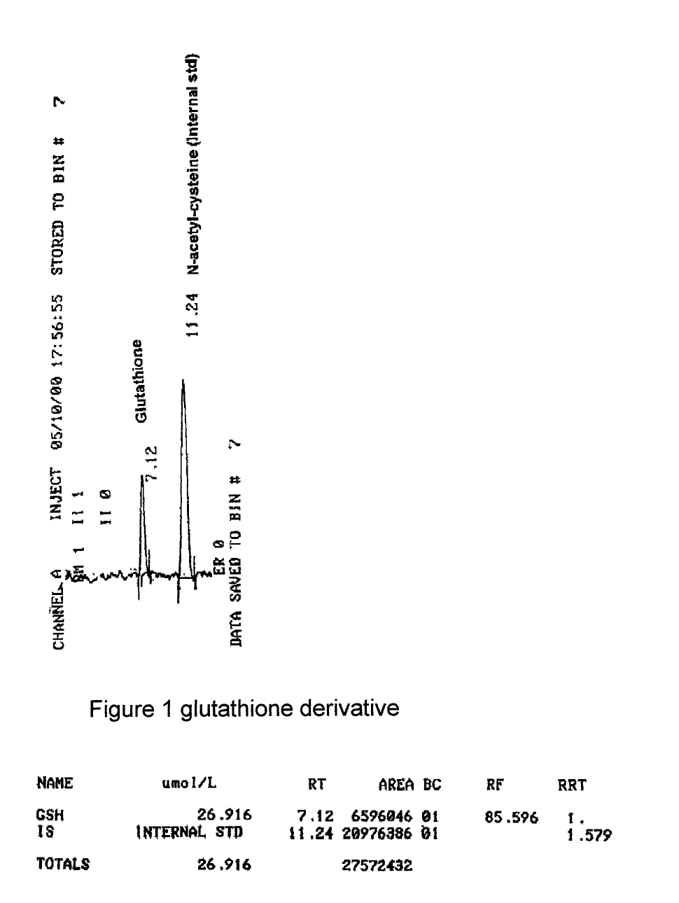

FIG. 2 is an HPLC analysis of control glutathione.

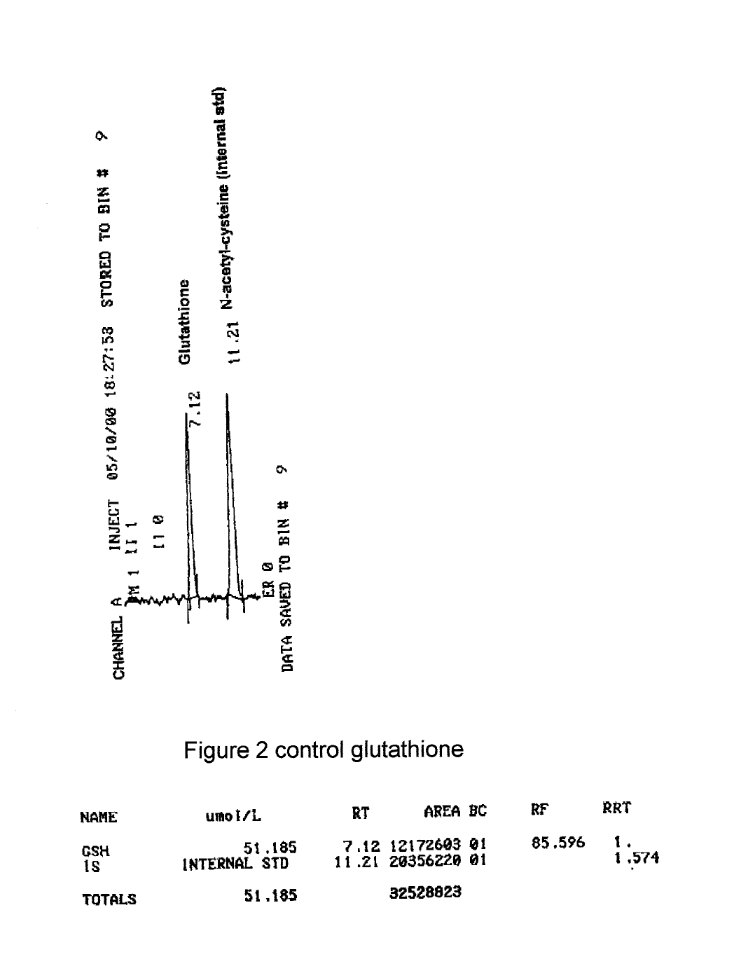

### DETAILED DESCRIPTION OF THE INVENTION:

As mentioned above, this invention is generally directed to new salts of glutathione. Such new glutathione salts, when administered to a warm blooded animal in need thereof, have utility in the prevention or treatment of conditions enumerated above in warm blooded animals, including humans.

The term “treat” or “treatment” means that the symptoms associated with one or more conditions mentioned above are alleviated or reduced in severity or frequency and the term “prevent” means that subsequent occurrences of such symptoms are avoided or that the frequency between such occurrences is prolonged.

It has now surprisingly been found that salts of glutathione with chitosan have good characteristics that are such as to render them particularly suitable both for use in pharmaceutical formulations and for preparative applications. Owing to their simple conception and low costs, the procedures described in this invention easily lend themselves to working out methods of preparation on an industrial scale.

The examples given herein below illustrate the preparation of two salts of glutathione with chitosan. Only a few of the many possible embodiments that may be anticipated are shown by these examples which are intended to define, in a non-limiting sense, the scope encompassed by the invention.

These examples are given to illustrate the present invention, but not by way of limitation. Accordingly, the scope of this invention should be determined not by the embodiments illustrated, but rather by the appended claims and their legal equivalents.

### EXAMPLE 1

Glutathione (0.45 g) was stirred in water (40 ml) and chitosan (0.25 g, degree of deacetylation 80.1%) was added with stirring. The solution was stirred until dissolved. The solution was filtered and dried.

### EXAMPLE 2

Glutathione (0.45 g) was stirred in water (60 ml) and chitosan (0.50 g, degree of deacetylation 80.1%) was added with stirring. The solution was stirred until dissolved. The solution was filtered and dried.

Glutathione and chitosan are available commercially from Sigma Chemical Company, St. Louis, Mo.

I claim:

1. A composition containing glutathione (GSH, GSSH) or nitroso glutathione or glutathione monoalkyl ester with a polycation to obtain a water soluble salt.

2. A composition of claim 1, in which a polycation is a polycationic carbohydrate or a mixture thereof

3. A composition of claim 2 wherein the polycationic carbohydrate is selected from chitosan, water soluble chitosan derivative, or a salt thereof.

4. A composition of claim 1 in which a polycation is selected from a polyaminoacid, a polyamine, a polypeptide, a basic polymer, a quartinary ammonium compound or a mixtures thereof

5. A compostion of claim 1 with at least one pharmaceutically acceptable carrier.

6. A composition of claim 1 in which a glutathione monoalkyl ester is selected from glutathione monomethyl ester, monoethyl ester or monooctyl ester or a mixtures thereof

7. The method of claim 1, wherein the compound is admistered in a pharmaceutically acceptable form selected from the group consisting of a tablet, a capsule, a syrup, a dragee, a suspension, an elixir, a solution, a powder, granules, an emulsion, microspheres, nanospheres, lipid vesicles, polymeric vesicles, of an injectable.

8. The method as defined by claim 1, wherein the compound is administered in a form selected from the group consisting of ointinent, a cream, a milk, a salve, an impregnated pad, a gel, a spray, and a lotion.

9. The method of claim 1, which comprises topical administration of said compound.

10. The method of claim 1, which comprises systemic administration of said compound.

11. The method of claim 1, wherein the treated condition is low cellular concentration of glutathione.

12. The method of claim 1, wherein the treated condition is low cellular concentration of nitroso glutathione

13. The method of claim 1, wherein the treated condition is chronic or acute liver disease.

14. The method of claim 1, which is used to treat acute and chronic lung disease such as acute respiratory distress syndrome, asthma, chronic obstructive pulmonary disease, ideopathic pulmonary fibrosis, cystic fibrosis, neonatal lung damage,

15. The method of claim 1, which is used to treat conditions associated with a decrease immune response such as HIV/AIDS, cancer, chronic viral infections

16. The method of claim 1, which is used to treat neurological conditions such as Parkinson's disease, dementia, Alzheimer's disease, tardive dyskinesia, schizophrenia, Down's syndrome,

17. The method of claim 1, which is used to treat cardiovascular conditions such as atherosclerosis

18. The method of claim 1, which is used to treat diabetes and its complications

19. The method of claim 1, which is used to correct age related cellular glutathione depletion.
